SEQUENCE LISTING (11 sequences)

SEQ ID NO: 1 - DNA, 24 bp, Artificial Sequence
  Primer
tgtccagtccagtaatggttctgt

SEQ ID NO: 2 - DNA, 21 bp, Artificial Sequence
  Primer
aagcgagatatggtccggatt

SEQ ID NO: 3 - DNA, 33 bp, Artificial Sequence
  Probe sequence
acacctggagagaagctttcaacgagactaacc

SEQ ID NO: 4 - DNA, 17 bp, Artificial Sequence
  Primer
ggtagggccaacggcct

SEQ ID NO: 5 - DNA, 25 bp, Artificial Sequence
  Primer
ctgtcttggttgttgataagctgaa

SEQ ID NO: 6 - DNA, 23 bp, Artificial Sequence
  Probe sequence
atcgcggagccatggattgcact

SEQ ID NO: 7 - DNA, 19 bp, Artificial Sequence
  Primer
ccggtggaccacgaagagt

SEQ ID NO: 8 - DNA, 20 bp, Artificial Sequence
  Primer
gctcgcctcttccatgtctc

SEQ ID NO: 9 - DNA, 25 bp, Artificial Sequence
  Probe sequence
aacccgggacttggagaagcactgc

SEQ ID NO: 10 - DNA, 64 bp, Artificial Sequence
  Synthetically generated oligonucleotide
gatccccgcactgcagagacatggaattcaagagattccatgtctctgcagtgctttttggaaa

SEQ ID NO: 11 - DNA, 64 bp, Artificial Sequence
  Synthetically generated oligonucleotide
agcttttccaaaaagcactgcagagacatggaatctcttgaattccatgtctctgcagtgcggg

(19) United States
(12) Patent Application Publication
(10) Pub. No.: US 2007/0032464 A1
(43) Pub. Date: Feb. 8, 2007

(54) Methods of treating cancers

(76) Inventors: Shutsung Liao, Chicago, IL (US); Junichi Fukuchi, Chiba-shi (JP)

Correspondence Address: FISH & RICHARDSON PC, P.O. BOX 1022, MINNEAPOLIS, MN, 55440-1022, US

(21) Appl. No.: 11/246,889

(22) Filed: Oct. 7, 2005

Related U.S. Application Data
(60) Provisional application No. 60/617,321, filed on Oct. 8, 2004.

(51) Int. Cl. A61K 31/56

(52) U.S. Cl. 514/169

(57) ABSTRACT

This invention relates to methods for treating cancers.

### CROSS REFERENCE TO RELATED APPLICATIONS

This application claims the benefit of U.S. Provisional Application No. 60/617,321, filed on Oct. 8, 2004, which is incorporated by reference herein in its entirety.

### TECHNICAL FIELD

This invention relates to methods for treating cancers.

### BACKGROUND

Prostate cancer is the most commonly diagnosed malignancy and the second leading cause of cancer death among American men. In general, prostate tumors are initially dependent on androgen for growth, even after metastasis, and therefore can be treated effectively by androgen deprivation. Prostate tumors can reappear, typically after 1 to 3 years of endocrine therapy, as androgen-independent tumors. Androgen deprivation or antiandrogen therapies are generally ineffective against androgen-independent tumors.

The normal prostate produces and secretes a relatively significant amount of cholesterol in prostatic fluid. In benign prostatic hypertrophy and prostatic adenocarcinorma, the levels of tissue and secreted cholesterol are two to ten fold higher than in healthy prostate. It has also been reported that sterol response element binding proteins (SREBPs), transcriptional regulators that control the metabolic pathway of lipogenesis and cholesterol, are activated in androgen-independent tumors.

Liver X receptors (LXRs), e.g., LXRα and LXRβ, are nuclear receptors, which are believed to function as central transcriptional regulators for lipid homeostasis. LXRs are believed to function as heterodimers with retinoid X receptors (RXRs), and these dimers can be activated by ligands for either receptor. LXRα is expressed at relatively high levels in liver, intestine, adipose tissue and macrophages, whereas LXRβ is expressed ubiquitously and has been dubbed the ubiquitous receptor (UR). LXR response elements in LXR-target genes are direct repeats of the consensus AGGTCA separated by four nucleotides. Since both LXRs in macrophages control the cholesterol efflux pathway through the regulation of target genes including ATP-binding cassette A1 (ABCA1) and apolipoprotein E, synthetic LXRs agonists have been developed as anti-atherogenic drugs.

### SUMMARY

In one aspect, this invention relates to a method for treating cancer (e.g., a cancer, which is associated with UR expression, e.g., breast cancer, colon cancer, prostate cancer, and leukemia), the method includes administering to a subject (e.g., a subject in need thereof) an effective amount of a Liver X Receptor agonist having formula (I):

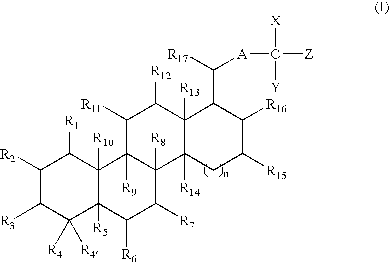

in which

each of R₁, R₂, R₃, R₄, R₄, R₅, R₆, R₇, R₁₁, R₁₂, R₁₅, R₁₆, and R₁₇, independently, is hydrogen, halo, alkyl, haloalkyl, hydroxy, amino, carboxyl, oxo, sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—; or R₃ and R₄ together, R₄ and R₅ together, R₅ and R₆ together, or R₆ and R₇ together are eliminated so that a C═C bond is formed between the carbons to which they are attached;

each of R₈, R₉, R₁₀, R₁₃, and R₁₄, independently, is hydrogen, halo, alkyl, haloalkyl, hydroxyalkyl, alkoxy, hydroxy, or amino;

n is 0, 1, or 2;

A is alkylene, alkenylene, or alkynylene; and

each of X, Y, and Z, independently, is alkyl, haloalkyl, —OR′, —SR′, —NR′R″, —N(OR′)R″, or —N(SR′)R″; or X and Y together are ═O, ═S, or ═NR′;

wherein each of R′ and R″, independently, is hydrogen, alkyl, or haloalkyl; or a salt (e.g., a pharmaceutically acceptable salt) thereof.

In another aspect, this invention also relates generally to inhibiting the proliferation of cancer cells with compounds having any one of the formulae described herein. In some embodiments, the methods can include in vitro methods, e.g., contacting a cell culture (e.g., representing one or more cancer cell lines) or a cancerous tissue (e.g., having one or more types of tumors) with a compound having any one of the formulae described herein. In other embodiments, the methods can include in vivo methods, e.g., administering a compound having having any one of the formulae described herein to a subject (e.g., a subject in need thereof, e.g., a mammal, e.g., a human).

Embodiments can include one or more of the following features.

The cancer can be a sex hormone-dependent cancer.

The sex hormone-dependent cancer can be prostate cancer. In some embodiments, the prostate cancer can be an androgen-dependent prostate cancer. In some embodiments, the prostate cancer can be resistant to androgen deprivation and/or antiandrogen therapies, (e.g., an androgen-independent prostate cancer, e.g., a hormone-refractory prostate cancer).

The subject can have at least one prostate cancer tumor that is resistant to androgen deprivation and/or antiandrogen therapies, e.g., an androgen-independent prostate cancer tumor. In some embodiments, the subject can further be substantially free of androgen-dependent prostate cancer tumors.

The sex hormone-dependent cancer can be breast cancer.

The Liver X receptor agonist can be orally administered.

The Liver X receptor can be LXRα or LXRβ.

Each of R₅ and R₆, independently, can be hydrogen, alkyl, haloalkyl, hydroxy, or amino.

R₅ can be H; and R₆ can be hydroxy.

X and Y together can be ═O or ═S; and Z can be —OR′, —SR′, —NR′R″, —N(OR′)R″, or —N(SR′)R″.

X and Y together can be ═O; and Z can be —NR′R″, —N(OR′)R″, or —N(SR′)R″.

Each of R₁, R₂, R₃, R₄, R_{4′}, R₇, R₈, R₉, R₁₁, R₁₂, R₁₄, R₁₅, R₁₆, and R₁₇, independently, can be hydrogen, halo, alkyl, haloalkyl, hydroxy, or amino; each of R₁₀ and R₁₃, independently, can be hydrogen, alkyl, or haloalkyl; n can be 0; and A can be alkylene.

Each of R₁, R₂, R₄, R₄′, R₇, R₈, R₉, R₁₁, R₁₂, R₁₄, R₁₅, R₁₆, and R₁₇, independently, can be hydrogen; R₃ is hydroxy; each of R₁₀ and R₁₃, independently, can be alkyl; and A can be alkylene.

Each of X, Y, and Z, independently, can be alkyl, haloalkyl, —OR′, or —SR′.

R₅ and R₆ together can be eliminated so that a C═C bond is formed between the carbons to which R₅ and R₆ are attached.

Each of R₁, R₂, R₄, R_{4′}, R₇, R₈, R₉, R₁₁, R₁₂, R₁₄, R₁₅, R₁₆, and R₁₇, independently, can be hydrogen, halo, alkyl, haloalkyl, hydroxy, or amino; R₃ can be hydroxy; each of R₁₀ and R₁₃, independently, can be alkyl; n can be 0; and A can be alkylene.

R₃ can be hydroxy; and each of R₁₀ and R₁₃, independently, can be alkyl.

Each of X, Y, and Z, independently, can be alkyl, haloalkyl, —OR′, —SR′, —NR′R″, —N(OR′)R″, or —N(SR′)R″.

Each of X, Y, and Z, independently, can be alkyl, haloalkyl, —OR′, or —SR′.

Each of R₁, R₂, R₄, R_{4′}, R₅, R₇, R₈, R₉, R₁₁, R₁₂, R₁₄, R₁₅, R₁₆, and R₁₇, independently, can be hydrogen; each of R₃ and R₆, independently, can be hydrogen or hydroxy; each of R₁₀ and R₁₃, independently, can be alkyl.

The compound can be:

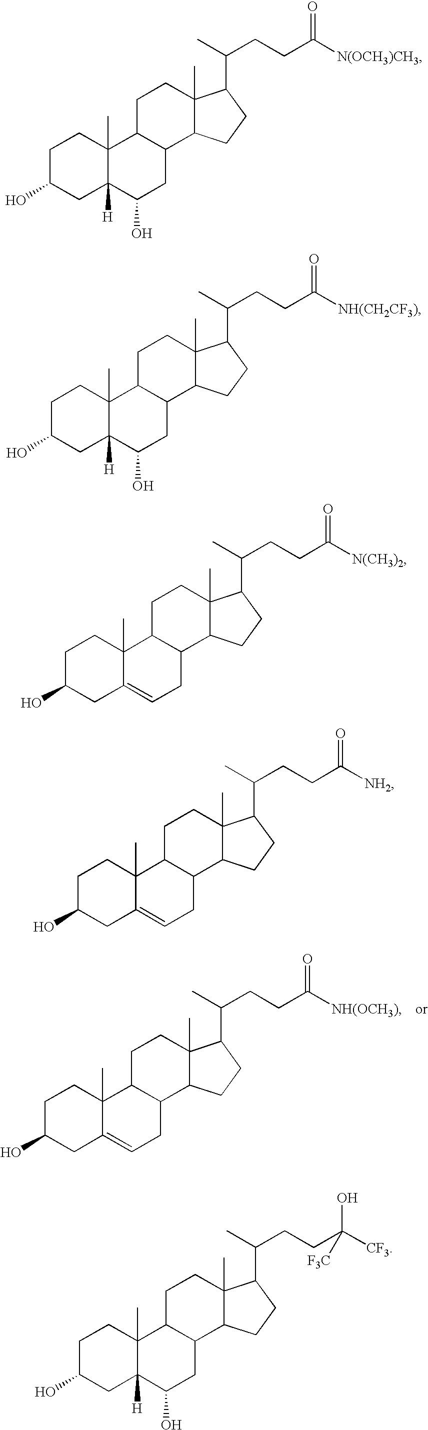

Each of X, Y, and Z can be, independently, haloalkyl or OR′ (e.g., two of X, Y, and Z can be, independently, haloalkyl, and the other can be OR′; e.g., two of X, Y, and Z are, independently, haloalkyl, and the other can be OH; e.g., two of X, Y, and Z can be CF₃, and the other can be hydroxy.

R₁, R₂, R₃, R₄, R₅, R₆, R₇, R₁₁, R₁₂, R₁₅, R₁₆, and R₁₇ can be independently hydrogen, halo, alkyl, haloalkyl, hydroxy, amino, carboxyl, oxo, sulfonic acid, or alkyl that is optionally substituted at one or more positions with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—; R^{4′} can be hydrogen; each of R₈, R₉, R₁₀, R₁₃, and R₁₄ can be, independently, hydrogen, halo, alkyl, haloalkyl, hydroxyalkyl, alkoxy, hydroxy, or amino; n can be 0, 1, or 2; A can be alkylene, alkenylene, or alkynylene; X, Y, and Z can be independently alkyl, haloalkyl, —OR′, —SR′, —NR′R″, —N(OR′)R″, or —N(SR′)R″; or X and Y together can be ═O, ═S, or ═NR′; and R′ and R″, can be independently hydrogen, alkyl, or haloalkyl; or a salt, an ester, an amide, an enantiomer, an isomer, a tautomer, a polymorph, a prodrug, or a derivative thereof.

R₁, R₂, R₄, R₇, R₈, R₉, R₁₁, R₁₂, R₁₄, R₁₅, and R₁₆ can be independently hydrogen; R₁₀, R₁₃, and R₂₀ can be independently alkyl; n can be 0; and A can be alkylene.

R₅ can be hydrogen; and R₃ and R₆ can be hydroxy.

R₅ can be beta-hydrogen; and R₃ and R₆ can be alpha-hydroxy.

X, Y, and Z, can be independently alkyl, haloalkyl, —OR′, or —SR′.

The compound can be:

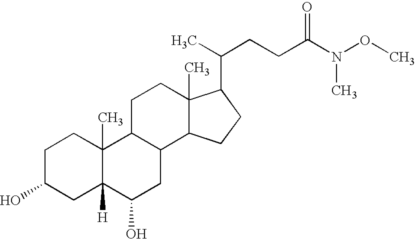

The compound can be:

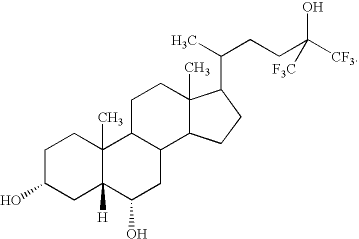

The method can include a compound having any one of the formulae described herein along with a pharmaceutically acceptable carrier or adjuvant.

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In another aspect, this invention also features compounds used in the above methods (e.g., a compound having any one of the formulae described herein).

In a further aspect, this invention also relates to methods of making compounds described herein. Alternatively, the method includes taking any one of the intermediate compounds described herein and reacting it with one or more chemical reagents in one or more steps to produce a compound described herein.

In one aspect, this invention relates to a packaged product. The packaged product includes a container, one of the aforementioned compounds in the container, and a legend (e.g., a label or an insert) associated with the container and indicating administration of the compound for treatment of any of the cancers described herein.

In another aspect, the invention relates to a compound (including a pharmaceutically acceptable salt thereof) of any of the formulae delineated herein, or a composition comprising a compound (including a pharmaceutically acceptable salt thereof) of any of the formulae delineated herein. In some embodiments, the composition can further include a pharmaceutically acceptable adjuvant, carrier or diluent and/or an additional therapeutic agent.

The term “mammal” includes organisms, which include mice, rats, cows, sheep, pigs, rabbits, goats, and horses, monkeys, dogs, cats, and humans.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/Kg to about 1000 mg/Kg, (e.g., from about 0.1 to about 100 mg/Kg, from about 1 to about 100 mg/Kg). In certain embodiments, the dosage can be about 10 mg/Kg daily. Effective doses will also vary depending on route of administration, as well as the possibility of co-usage with other agents.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₂₀ alkyl indicates that the group may have from 1 to 20 (inclusive) carbon atoms in it. Any atom can be substituted. Examples of alkyl groups include without limitation methyl, ethyl, and tert-butyl.

The term “cycloalkyl” refers to saturated monocyclic, bicyclic, tricyclic, or other polycyclic hydrocarbon groups. Any atom can be substituted, e.g., by one or more substituents. Cycloalkyl groups can contain fused rings. Fused rings are rings that share a common carbon atom. Cycloalkyl moieties can include, e.g., cyclopropyl, cyclohexyl, methylcyclohexyl (the point of attachment to another moiety can be either the methyl group or a cyclohexyl ring carbon), adamantyl, and norbomyl.

The term “haloalkyl” refers to an alkyl group in which at least one hydrogen atom is replaced by halo. In some embodiments, more than one hydrogen atom (2, 3, 4, 5, 6, 7, 8, 9, 10, 11, 12, 13, 14, 15, 16, 17, 18, 19, 20, 21, 22, 23, 24, 25, 26, etc. hydrogen atoms) on an alkyl group can be replaced by more than one halogens (e.g., 2, 3, 4, 5, 6, 7, 8, 9, 10, 11, 12, 13, 14, 15, 16, 17, 18, 19, 20, 21, 22, 23, 24, 25, 26, etc. hydrogen atoms), which can be the same or different. “Haloalkyl” also includes alkyl moieties in which all hydrogens have been replaced by halo (e.g., perhaloalkyl, such as trifluoromethyl).

The term “aralkyl” refers to an alkyl moiety in which an alkyl hydrogen atom is replaced by an aryl group. Aralkyl includes groups in which more than one hydrogen atom on an alkyl moiety has been replaced by an aryl group. Any ring or chain atom can be substituted e.g., by one or more substituents. Examples of “aralkyl” include without limitation benzyl, 2-phenylethyl, 3-phenylpropyl, benzhydryl, and trityl groups.

The term “heteroaralkyl” refers to an alkyl moiety in which an alkyl hydrogen atom is replaced by a heteroaryl group. Heteroaralkyl includes groups in which more than one hydrogen atom on an alkyl moiety has been replaced by a heteroaryl group. Any ring or chain atom can be substituted e.g., by one or more substituents. Heteroaralkyl can include, for example, 2-pyridylethyl.

The term “alkenyl” refers to a straight or branched hydrocarbon chain containing 2-20 carbon atoms and having one or more double bonds. Any atom can be substituted, e.g., by one or more substituents. Alkenyl groups can include, e.g., allyl, propenyl, 2-butenyl, 3-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be substituted, e.g., by one or more substituents. Alkynyl groups can include, e.g., ethynyl, propargyl, and 3-hexynyl. One of the triple bond carbons can optionally be the point of attachment of the alkynyl substituent.

The terms alkylene, alkenylene, and alkynylene refer to divalent alkyl, alkenyl, and alkynyl groups, respectively.

The term “alkoxy” refers to an —O-alkyl radical. The term “mercapto” refers to an SH radical. The term “thioalkoxy” refers to an —S-alkyl radical. The term aryloxy refers to an —O-aryl radical. The term thioaryloxy refers to an —S-aryl radical.

The term “heterocyclyl” refers to a monocyclic, bicyclic, tricyclic or other polycyclic ring system having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). The heteroatom can optionally be the point of attachment of the heterocyclyl substituent. Any atom can be substituted, e.g., by one or more substituents. The heterocyclyl groups can contain fused rings. Fused rings are rings that share a common carbon atom. Heterocyclyl groups can include, e.g., tetrahydrofuranyl, tetrahydropyranyl, piperidinyl, morpholino, pyrrolinyl, and pyrrolidinyl.

The term “cycloalkenyl” refers to partially unsaturated monocyclic, bicyclic, tricyclic, or other polycyclic hydrocarbon groups. The unsaturated carbon can optionally be the point of attachment of the cycloalkenyl substituent. Any atom can be substituted e.g., by one or more substituents. The cycloalkenyl groups can contain fused rings. Fused rings are rings that share a common carbon atom. Cycloalkenyl moieties can include, e.g., cyclohexenyl, cyclohexadienyl, norbomenyl, or cyclooctenyl.

The term “heterocycloalkenyl” refers to partially unsaturated monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). The unsaturated carbon or the heteroatom can optionally be the point of attachment of the heterocycloalkenyl substituent. Any atom can be substituted, e.g., by one or more substituents. The heterocycloalkenyl groups can contain fused rings. Fused rings are rings that share a common carbon atom. Heterocycloalkenyl groups can include, e.g., tetrahydropyridyl, and dihydropyranyl.

The term “aryl” refers to a monocyclic, bicyclic, or tricyclic aromatic moiety and can contain fused rings. Fused rings are rings that share a common carbon atom. Typical examples of aryl include phenyl, naphthyl, and anthracenyl.

The term “heteroaryl” refers to an aromatic monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (e.g., carbon atoms and 1-4, 1-8, or 1-10 heteroatoms of N, O, or S if monocyclic, bicyclic, or tricyclic, respectively). Any atom can be substituted, e.g., by one or more substituents. Heteroaryl groups can contain fused rings. Fused rings are rings that share a common carbon atom. Heteroaryl groups include pyridyl, thienyl, furanyl, imidazolyl, and pyrrolyl.

The term “oxo” refers to an oxygen atom, which forms a carbonyl when attached to carbon, an N-oxide when attached to nitrogen, and a sulfoxide or sulfone when attached to sulfur.

The term “substituents” refers to a group “substituted” on, e.g., an alkyl, cycloalkyl, alkenyl, alkynyl, aralkyl, heteroaralkyl, heterocyclyl, heterocycloalkenyl, cycloalkenyl, aryl, or heteroaryl group at any atom of that group. In one aspect, the substituents on a group are independently any one single, or any subset of the aforementioned substituents. In another aspect, a substituent may itself be substituted with any one of the above substituents.

The details of one or more embodiments of the invention are set forth in the accompanying drawings and the description below. Other features and advantages of the invention will be apparent from the description and from the claims.

### DETAILED DESCRIPTION

Compounds of this invention can be synthesized by methods well known in the art by using a suitable steroid as a starting material. More specifically, such a steroid possesses a substitutent at C-17 [the carbon to which R₁₇ is attached, see formula (I) above] that can be modified to contain a moiety defined by X, Y, and Z [also shown in formula (I)]. Examples include cholic acid, dehydrocholic acid, deoxycholic acid, lithocholic acid, ursodeoxycholic acid, hyocholic acid, hyodeoxycholic acid, and cholanoic acid. They are either commercially available or can be synthesized by methods described in the literature, e.g., Roda et al., F. Lipid Res., 1994, 35: 2268-2279; and Roda et al., Dig. Dis. Sci., 1987, 34: 24S-35S.

A compound of this invention that has an amide-containing substitutent at C-17 (i.e., X and Y together are ═O, and Z is amine) can be prepared by reacting a steroid having a carboxyl-containing substituent at C-17 with an amino-containing compound (such as dimethylamine, aniline, glycine, and phenylalanine). Similarly, a compound of this invention that has an ester-containing substitutent at C-17 (i.e., X and Y together are ═O, and Z is alkoxy) can be prepared by reacting a steroid having a carboxyl-containing substituent at C-17 with a hydroxyl-containing compound (such as ethanol and isopropanol). The amide- or ester-forming reaction can take place in any suitable solvents. If the reaction takes place in an aqueous solution, isolation of the steroid product for in vitro or in vivo screening assays may not be necessary.

A compound of this invention that has a carbonyl-containing substitutent at C-17 (i.e., X and Y together are ═O) can be converted, e.g., to a thiocarbonyl-containing compound of this invention (i.e., X and Y together are ═S) by reacting it with sulfur hydride, or to an imino-containing compound of this invention (i.e., X and Y together are ═NR) by reacting it with hydrazine. See Janssen et al. (Ed.), Organosulfur Chemistry; Wiley: New York, 1967, 219-240; and Patai et al. (Ed.), The Chemistry of the Carbon-Nitrogen Double Bond; Wiley: New York, 1970, 64-83 and 465-504, respectively.

Substituents at ring atoms other than C-17, if necessary, can further be modified by methods well known in the art. For instance, a hydroxyl substituent at C-3 can be converted to an ester substituent by reacting it with an acid such as acetic acid.

Due to the simplicity of the reaction, it can be easily automated. Isolation and quantification of the product can be done by thin-layer chromatography, high pressure liquid chromatography, gas chromatography, capillary electrophoresis, or other analytical and preparative procedures.

A compound that does not contain a carbonyl, thiocarbonyl, or imino group in the C-17 substituent can also be prepared by methods well known in the art. For instance, 3α,6α,24-trihydroxy-24,24-di(trifluoromethyl)-5β-cholane can be prepared according to the following scheme:

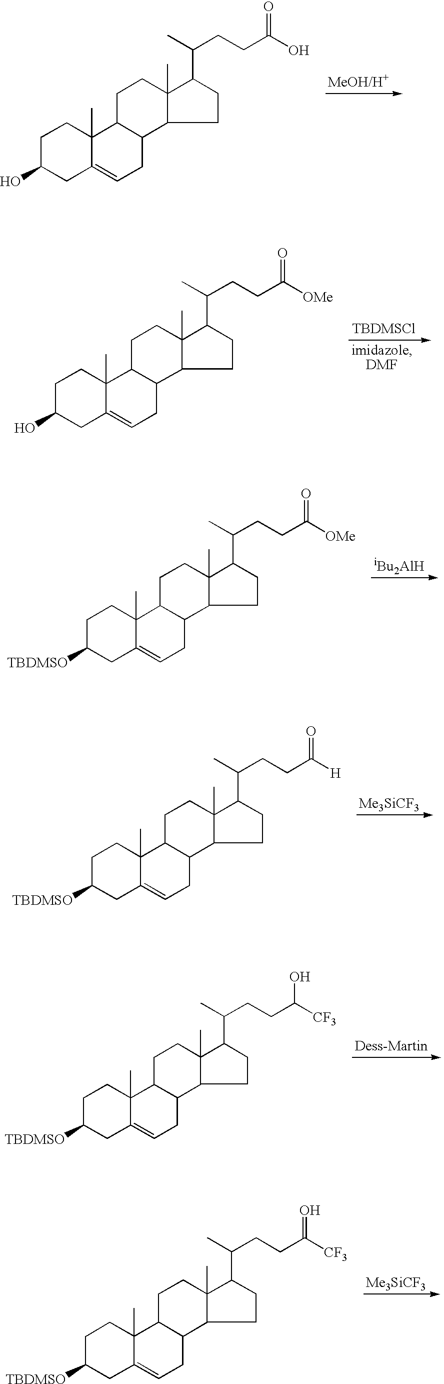

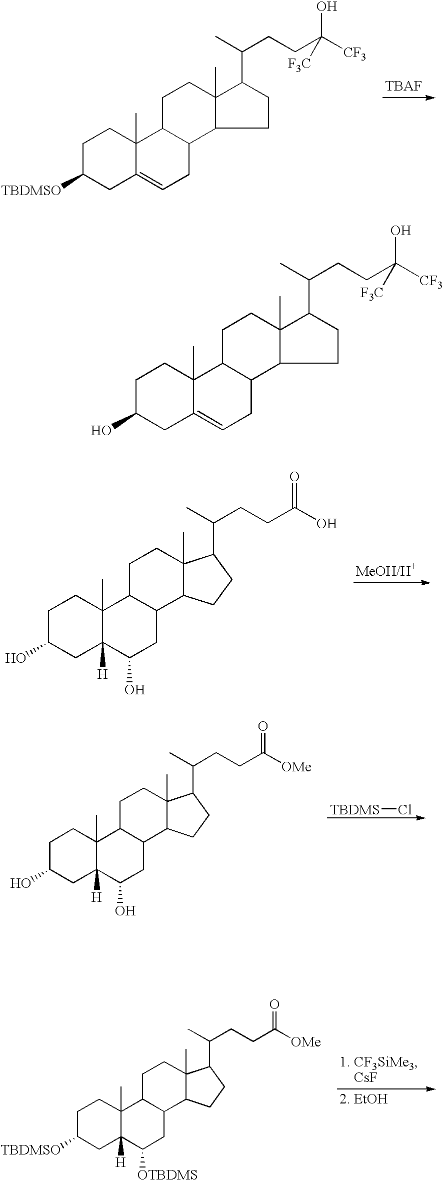

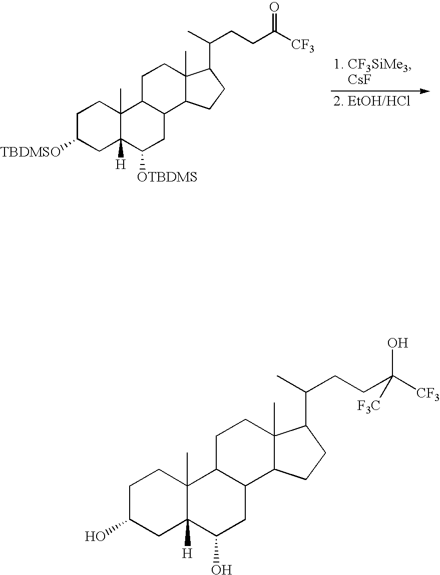

As shown in the above schemes, cholanoic acid is first reacted with methanol in the presence of an acid to afford its methyl ester, which is subsequently reacted with tert-butyldimethylsilyl chloride (TBDMSCl) for protection of the 3β-hydroxyl group. The protected methyl ester is then converted to an aldehyde by reacting with di(iso-butryl)alumina hydride, which is subsequently converted to an alcohol, α-substituted with trifluoromethyl, by reacting with trimethyl(trifluoromethyl)silane. The alcohol then undergoes the Dess-Martin reaction for conversion to a ketone. See Dess et al., J. Org. Chem., 1983, 38: 4155. The ketone is treated with trimethyl(trifluoromethyl)silane again to afford an alcohol, a-substituted with two trifluoromethyl groups. Finally, the disubstituted alcohol is deprotected by reacting it with tetrabutylammonium fluoride (TBAF) to afford 3α,6α,24-trihydroxy-24,24-di(trifluoromethyl)-5β-cholane.

The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quatemization of any basic nitrogen-containing groups of the compounds disclosed herein. Water or oil-soluble or dispersible products may be obtained by such quatemization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

An in vitro assay can be conducted to preliminarily screen a compound of this invention for its efficacy in agonizing LXRs and thus in treating an LXR-mediated disease. For instance, kidney cells are transfected with a luciferase reporter gene (which includes a human c-fos minimal promoter) and an LXR. After incubating the transfected cells with a compound to be tested, the activity of luciferase is measured to determine the transactivation extent of the reporter gene.

Compounds that show efficacy in the preliminary assay can be further evaluated in an animal study by a method also well known in the art. For example, a compound can be orally administered to mice fed with a cholesterol-containing diet. The efficacy of the compound can be determined by comparing cholesterol levels in various tissues of the treated mice with those in non-treated mice.

The compounds described herein can be used for treating cancer, e.g., cancers which are associated with UR expression, e.g., breast cancer, colon cancer, prostate cancer, and leukemia. In some embodiments, the cancer can be a sex hormone-dependent cancer (e.g., prostate cancer or breast cancer).

In some embodiments, the sex hormone-dependent cancer can be prostate cancer. In certain embodiments, the prostate cancer can be an androgen-dependent prostate cancer. In certain embodiments, the prostate cancer can be resistant to conventional androgen deprivation and/or antiandrogen therapies (e.g., an androgen-independent prostate cancer, e.g., a hormone-refractory prostate cancer). For example, a subject (e.g., a patient, e.g., a human patient) can have at least one prostate cancer tumor that is relatively resistant to androgen deprivation and/or antiandrogen therapies, e.g., an androgen-independent prostate cancer tumor. In some embodiments, the subject can further be substantially free of androgen-dependent prostate cancer tumors. Androgen-independent prostate cancer and hormone-refractory prostate cancer are described in, e.g., Kasamon, et al., Curr. Opin. Urol. 14: 185-193 (2004).

In one embodiment of the present invention, the compounds activate the liver X receptor alpha (that is, an liver X receptor alpha agonist). In another embodiment of the present invention, the compounds selectively activate the liver X receptor alpha (that is, a selective liver X receptor alpha agonist) relative to liver X receptor beta. In one embodiment, the compounds of the present invention have a selectivity ratio of liver X receptor alpha relative to liver X receptor beta of at least 2; in another embodiment have a selectivity ratio of at least 25; in another embodiment have a selectivity ratio of at least 50; in another embodiment have a selectivity ratio of at least 100, and in another embodiment have a selectivity ratio of at least 1,000. As used herein, the term liver X receptor agonist encompasses both a liver X receptor alpha agonist and a selective liver X receptor alpha agonist, unless the context in which it is used dictates otherwise.

Illustratively, agonists of liver X receptor alpha used in the treatment, prevention or reduction in the risk of developing cancer may activate the liver X receptor alpha activity through a variety of mechanisms. By way of example, the liver X receptor alpha agonist used in the methods described herein may activate the receptor directly by binding to the receptor, such as a ligand. While not wishing to be bound by theory, the use of a liver X receptor alpha selective activator can be advantageous in that they may increase the HDL cholesterol level, and/or decrease the LDL cholesterol level in serum or in the liver without increasing serum triglycerides levels.

In some embodiments, the compounds described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of one or more compounds of any of the formulae described herein). Alternatively, those agents may be part of a single dosage form, mixed together with the compounds of this invention in a single composition (e.g., simultaneously or at about the same with one or more compounds of any of the formulae described herein). When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. In some embodiments, the therapeutic agent can be an RXR agonist (e.g., LGD1069, Bexarotene, Tagretin). RXR agonists are described in, e.g., Lippman et al., Journal of Nutrition (2000) Supplement 479S-482S; and Staels J. Am. Acad. Dermatol. (2001) 45, S158-S167.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/Kg to about 2000 mg/Kg, (e.g., from about 0.01 mg/Kg to about 100 mg/kg, from about 0.1 mg/Kg to about 100 mg/Kg, 1 mg/kg to about 2000 mg/Kg, from about 1 mg/Kg to about 1000 mg/Kg, or from about 1 mg/kg to about 500 mg/kg; from about 1 mg/Kg to about 100 mg/Kg, from about 1 mg/Kg to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, New York, 537 (1970). In certain embodiments, the compositions are administered by oral administration. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable excipients, carriers, adjuvants or vehicles. In some cases, the pH of the formulation may be adjusted with pharmaceutically acceptable acids, bases or buffers to enhance the stability of the formulated compound or its delivery form. In some embodiments, solubilizing agents such as cyclodextrins, or other solubilizing agents well-known to those familiar with the art, can be utilized as pharmaceutical excipients for delivery of the therapeutic compounds.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution, isotonic sodium chloride solution, and 5% glucose. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil, sesame oil or castor oil, e.g., in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, gel seal, capsules, tablets, syrups, emulsions and aqueous suspensions, dispersions and solutions. In the case of tablets for oral use, carriers which are commonly used include starch, sugar bentonite, lactose and corn starch. Lubricating agents, such as magnesium stearate, are also typically added. Tablets may be formulated in accordance with the conventional procedure by compressing mixtures of the compound of this invention and a solid carrier, and a lubricant. The compounds of this invention can also be administered in a form of a hard shell tablet or a capsule containing a binder (e.g., lactose or mannitol) and a conventional filler. For oral administration in a capsule form, useful diluents include gelatin, cellulose derivatives, lactose and dried corn starch. When aqueous suspensions and/or emulsions are administered orally, the active ingredient may be suspended or dissolved in an oily phase is combined with emulsifying and/or suspending agents. In some embodiments, the vehicle for oral administration can be a pharmaceutically-acceptable oils, e.g., a natural oil, such as olive oil, sesame oil or castor oil. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing,) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

The invention will be further described in the following examples. It should be understood that these examples are for illustrative purposes only and are not to be construed as limiting this invention in any manner.

### EXAMPLES

### General

A monoclonal anti-p27 antibody is obtained from Transduction Laboratories (Lexington, Ky.). Polyclonal anti-Skp2 and anti-p21 goat IgGs are obtained from Santa Cruz Biotechnology (Santa Cruz, Calif.). A monoclonal anti-actin antibody is from Chemicon (Temecula, Calif.). A monoclonal anti-c-Myc antibody 9E10 is prepared from hybridoma obtained from the American Type Culture Collection (ATCC) (Manassas, Va.). Human prostate cancer DU-145, PC-3, human breast cancer MCF-7 and MDA-MB435S cells are obtained from ATCC and maintained in Dulbecco's modified Eagle medium supplemented with 10% fetal bovine serum.

Data are presented as the mean±standard deviation or standard error of three experiments or are representative of experiments repeated at least three times.

### Example 1

### Inhibition of Human Prostate Cancer Cell Growth

To determine whether the LXR agonists described herein inhibit human prostate cancer growth, androgen-dependent LNCaP 104-S cells and androgen-independent LNCaP 104-R1 cells are treated with a candidate compound having formula (I).

Androgen-dependent LNCaP 104-S cells and androgen-independent LNCaP 104-R1 cells are maintained and cultured as described in, e.g., Kokontis J, Takakura K, Hay N, and Liao S. “Increased androgen receptor activity and altered c-myc expression in prostate cancer cells after long-term androgen deprivation,” Cancer Res. 1994; 54: 1566-73; and Kokontis J M, Hay N, and Liao S. “Progression of LNCaP prostate tumor cells during androgen deprivation: hormone-independent growth, repression of proliferation by androgen, and role for p27Kip1 in androgen-induced cell cycle arrest,” Mol Endocrinol 1998; 12: 941-53.

The 104-S and 104-R1 cells are grown for 4 days in the presence of a candiate compound at concentrations of 1 μM, 2.5 μM, 5. μM, and 10 μM. Cell number is analyzed by measuring DNA content with the fluorescent dye Hoechst 33258 (SIGMA, St. Louis, Mo.) as described in, e.g., Rago R, Mitchen J, and Wilding G. “DNA fluorometric assay in 96-well tissue culture plates using Hoechst 33258 after cell lysis by freezing in distilled water,” Anal Biochem. 1990; 191: 31-4. The growth data is presented as per cent of vehicle control.

### Example 2

### Expression of LXR Target Genes

The expression of LXR-target genes is analyzed by real-time quantitative PCR. Total RNA is isolated using the TRIZOL Reagent (Invitrogen, Carlsbad, Calif.) and is treated with DNase I (DNA-free, Ambion, Austin, Tex.). Reverse transcription is performed with random hexamers and Moloney murine leukemia virus reverse transcriptase (Omniscript, QIAGEN, Valencia, Calif.). The TaqMan primer/probe is designed using Primer Express (Applied Biosystems, Foster City, Calif.). The 5′-end of the probe is labeled with the reporter-fluorescent dye, FAM. The 3′-end of probe is labeled with the quencher dye, TAMRA. The sequences of primers and probes are as follows: ABCA1 primers, 5′-TGTCCAGTCCAGTAATGGTTCTGT-3′ and 5′-AAGCGAGATATGGTCCGGATT-3′, ABCA1 probe 5′-ACACCTGGAGAGAAGCTTTCAACGAGACTAACC-3′; SREBP-1c primers, 5′-GGTAGGGCCAACGGCCT-3′ and 5′-CTGTCTTGGTTGTTGATAAGCTGAA-3′, SREBP-1c probe, 5′-ATCGCGGAGCCATGGATTGCACT-3′; p27 primers, 5′-CCGGTGGACCACGAAGAGT-3′ and 5′-GCTCGCCTCTTCCATGTCTC-3′, p27 probe, 5′-AACCCGGGACTTGGAGAAGCACTGC-3′, respectively. Real-time PCR is performed on an ABI PRISM 7700 system (Applied Biosystems) using the QuantiTect Probe RT-PCR protocol (QIAGEN). The Ribosomal RNA Control Kit (Applied Biosystems) is used to normalize transcript levels between samples.

### Example 3

### Effect of LXR Agonists on Cell Cycle Distribution

The effect of LXR receptor agonists on cell cycle distribution in the LNCaP sublines 104-S and 104-R1 is examined using flow cytometry of propidium iodide-stained cells. Cells are seeded at 5×10⁵ cells in 6 cm dishes. Cells are collected and fixed in 70% ethanol/30% phosphate buffered saline (PBS) overnight at −20° C. Fixed cells are washed with PBS, treated with 0.1 mg/ml RNase A in PBS for 30 minutes and then suspended in 50 μg/ml propidium iodide in PBS. Cell cycle profiles and distributions are determined using a BD Facscan flow cytometer (BD Biosciences, San Jose, Calif.). Cell cycle distribution is analyzed using ModFit LT software (Verity Software House, Topsham, Me.).

Since the expression level of the cell cycle dependent kinase inhibitor p27 is increased when LNCaP cells are arrested and in G1 phase (see, e.g., Kokontis J M, Hay N, and Liao S. “Progression of LNCaP prostate tumor cells during androgen deprivation: hormone-independent growth, repression of proliferation by androgen, and role for p27Kipl in androgen-induced cell cycle arrest,” Mol. Endocrinol. 1998; 12: 941-53), Western blotting can be performed to examine the effect of LXR receptor agonists on p27 expression. Protein extracts are prepared by lysing PBS-washed cells on the dish with Laemmli gel loading buffer without bromophenol blue dye. Protein concentration is determined with the Bradford reagent (Bio-Rad Laboratories, Hercules, Calif.) using a bovine serum albumin standard. Proteins are separated on 6% polyacrylamide gels containing SDS. Electrophoresis and blotting are performed as described in, e.g., Kokontis J M, Hay N, and Liao S. “Progression of LNCaP prostate tumor cells during androgen deprivation: hormone-independent growth, repression of proliferation by androgen, and role for p27Kipl in androgen-induced cell cycle arrest,” Mol. Endocrinol. 1998; 12: 941-53. Measurement of actin expression is used as a loading control.

Other molecules believed to be involved in LNCaP cell proliferation can also be analyzed by Western analysis (see e.g., Kokontis J, Takakura K, Hay N, and Liao S. “Increased androgen receptor activity and altered c-myc expression in prostate cancer cells after long-term androgen deprivation,” Cancer Res. 1994; 54: 1566-73; and Kokontis J M, Hay N, and Liao S. “Progression of LNCaP prostate tumor cells during androgen deprivation: hormone-independent growth, repression of proliferation by androgen, and role for p27Kip1 in androgen-induced cell cycle arrest,” Mol. Endocrinol. 1998; 12: 941-53).

To demonstrate that the level of p27 is functionally involved in LXR receptor agonist-induced cell cycle arrest, p27-knockdown 104-R1 cells are generated using an expression plasmid generating RNAi for p27. The RNAi sequence is designed by using the AA scanning program from OligoEngine (Seattle, Wash.). DNA coding for an RNAi for human p27 is prepared using the following oligonucleotides: 5′-GATCCCCGCACTGCAGAGACATGGAATTCAAGAGATTCCATGTCTCTGCAGT GCTTTTTGGAAA-3′ and 5′-AGCTTTTCCAAAAAGCACTGCAGAGACATGGAATCTCTTGAATTCCATGTCTC TGCAGTGCGGG-3′. These 64-mer oligonucleotides are annealed and ligated into the pH1RP vector (see, e.g., Fukuchi J, Hiipakka R A, Kokontis J M, Nishimura K, Igarashi K, and Liao S. “TATA-binding protein-associated factor 7 regulates polyamine transport activity and polyamine analog-induced apoptosis,” J. Biol. Chem. 2004; 279: 29921-9). The p27-RNAi expression plasmid is stably transfected into 104-S cells using Effectene (QIAGEN) and selection for G418 resistance.

### Example 4

### Inhibition of Breast and Other Prostate Cancer Cell Growth

The effect of LXR receptor agonists on the growth of various breast and other prostate cancer cell lines can also be determined. These cell lines can include: human prostate cancer PC-3 cells, breast cancer MCF-7 and MDA-MB435S cells, and human prostate cancer LNCaP and DU-145 cells.

Using retroviral infection, human LXRα in MDA-MB435S cells are ectopically expressed. Ectopic expression of LXRα is achieved by infecting MDA-MB534S cells with pLNCX2 retrovirus (Clonetech, Palo Alto, Calif.) carrying the human LXRα cDNA (see, e.g., Janowski B A, Willy P J, Devi T R, Falck J R, and Mangelsdorf D J. “An oxysterol signalling pathway mediated by the nuclear receptor LXR alpha,” Nature 1996; 383: 728-31). Retrovirus is generated using the Phoenix-ampho packaging cell line (G. Nolan, Stanford University).

### Example 5

### Athymic Nude Mice Study

To determine whether LXR receptor agonists have anti-proliferation effects in vivo, a candidate LXR receptor agonist is tested against LNCaP 104-S xenografts in athymic nude mice. Six to eight week old male BALB/c nu/nu mice (NCI-Frederick, Frederick, Md.) are injected subcutaneously (see, e.g., Umekita Y, Hiipakka R A, Kokontis J M, and Liao S. “Human prostate tumor growth in athymic mice: inhibition by androgens and stimulation by finasteride,” Proc. Natl. Acad. Sci. U. S. A. 1996; 93: 11802-7) with 106 LNCaP 104-S cells suspended in 0.25 ml of Matrigel (BD Bioscience, Bedford, Md.). Tumors are measured weekly using a caliper and their volumes are calculated using the formula length×width×height×0.52. In some embodiments, the initial tumor volumes can be about 90 mm³ prior to treatment. The candidate LXR receptor agonist is administered via daily oral gavage using sesame oil vehicle at a dose of about 10 mg/kg body weight per day.

### Example 6

### Synthesis of Compounds of This Invention

3α,6α,24-trihydroxy-24,24-di(trifluoromethyl)-5β-cholane [Compound (1)] was synthesized by the method described above.

3α,6α-dihydroxy-5β-cholanoic acid-N-methyl-N-methoxy-24-amide [Compound (2)], 2,2,2-trifluoroethyl-3α,6α-dihydroxy-5β-cholanoic acid 24-amide [Compound (3)], 24-cholesten-amide [Compound (4)], N,N-dimethyl-24-cholesten-amide [Compound (5)], and N-methoxy-24-cholesten-amide [Compound (6)] were synthesized by the following method:

A steroid 24-carboxylic acid (Sigma, St. Louis, Mo.), an amine, diethyl cyanophosphonate (Aldrich, Milwaukee, Wis.), and triethylamine were dissolved in dimethylformamide. The solution was stirred at 20-70° C. for 12-16 hours, quenched with ice, and then extracted with ethyl acetate. The ethyl acetate extract thus obtained was washed subsequently with a 1.0 N HCl solution and with a 1.0 N NaOH solution, and then dried over anhydrous sodium sulfate. The crude product was obtained after removal of ethyl acetate and was purified using standard silica chromatography if necessary.

### Example 7

### Synthesis of N-methyl-N-methoxy-3α,6α-dihydroxy-5β-cholanoic acid-24-amide(hypocholamide)

Into 300 mL 1,4-dioxane on ice was added 50 g of 3α,6α-dihydroxy-5β-cholanoic acid. Into the 1,4-dioxane solution was then dropwise added 15 mL ethylchloroformate the stirring, followed by addition of 30 mL triethylamine. The temperature of the solution thus obtained was raised to 20° C. and then stirred for 30 minutes. After that, 15 g of N,O-dimethylhydroxyarnine hydrochloride was added into the solution, which was then stirred for another 30 minutes before 20 mL of 1 N NaOH solution was added to it. The solution was stirred for additional 16 hours. For work-up, the reaction solution was poured into 2000 mL 1N HCl on ice, followed by extraction with ethylacetate. The ethylacetate layer was washed in sequence, with 1N HCl, water, 1N NaOH, and water; and was then dried over anhydrous MaSO₄. The ethylacetate solvent was removed under reduced pressure. The residue was purified with a silica gel column to give pure hypocholamide in white foam at a 75% yield.

¹H NMR (CDCl₃): 4.07 (m, 1H); 3.70 (s, 3H); 3.62 (m, 1H); 3.18 (s, 3H); 1.05-2.50 (m, 26H); 0.92-0.95 (m, 3H); 0.91 (s, 3H); 0.65 (s, 3H).

¹³C NMR: 171.0, 71.6, 68.1, 61.2, 56.1, 55.4, 48.5, 42.8, 39.9, 39.8, 35.9, 35.5, 35.0, 34.8, 30.6, 30.2, 29.2, 28.8, 28.1, 24.2, 23.5, 20.7, 18.4, 12.0, 8.0.

### Example 8

### Synthesis of 3α,6α,24-trihydroxy-5β-24,24-di(trifluoromethyl)-cholestane(hypocholaride)

19.2 g of 3α,6α-dihydroxy-cholic acid was dissolved in 200 mL anhydrous methanol. To the solution was then added 0.4 g of p-toluenesulfonic acid. After stirring at room temperature overnight, the methanol solvent was removed under reduced pressure to give a crude product (i.e., 3α,6α-dihydroxy-cholic acid methyl ester) in white foam.

Crude 3α,6a-dihydroxy-cholic acid methyl ester was then dissolved in 90 mL dimethylforamide (DMF). Into the DMF solution thus obtained was added 21.3 g TBDMS-Cl (1.5 eq.) and 24.0 g (3.75 eq.). The mixture was subsequently heated at 90° C. for 1 hour for protection of the 3α,6α hydroxy groups. The DMF solvent was subsequently removed under vacuum and the residue was added into ethyl ether and washed with sodium hydrogen carbonate and brine sequentially. After being dried over anhydrous sodium sulfate, ethyl ether was removed under reduced pressure. The residue was purified by a silica gel column to give a pure hydroxy-protected product in white foam at a 95% yield.

6.5 g of the hydroxy-protected product thus obtained was first dissolved in 60 mL glycol dimethyl ether. To the solution thus obtained were then added 1.5 mL trimethyl(trifluoromethyl)silane and a catalytic amount of CsF at room temperature. After stirring overnight, ethanol was added to the solution. The solution was then stirred at room temperature for 1 hour before all the solvents were removed under reduced pressure to give crude product (i.e., trifluoromethylketone).

The crude trifluoromethylketone product was dissolved in 60 mL glycol dimethyl ether. Into the solution were then added 1.5 mL trimethyl(trifluoromethyl)silane and a catalytic amount of CsF at room temperature. After the solution was stirred overnight, 3 mL ethanol was added to it. The solution was then further stirred at room temperature for 1 hour before all the solvents were removed under reduced pressure. The residue thus obtained was dissolved in a mixture of 100 mL ethanol and 3 mL concentrated hydrogen chloride. The ethanol solution was stirred for 1 hour, and the solvent was then removed under reduced pressure. The residue was subject to column purification to give the product (i.e., hypocholaride) as a white solid.

¹H NMR (CD₃OD): 4.00 (m, 1H); 3.50 (m, 1H); 0.92˜1.89 (m, 32 H); 0.67 (s 3H).

¹³C NMR: 123.6 (dd, 280 Hz); 76.0 (m); 70.9; 67.1, 56.1, 55.7, 42.5, 39.8, 39.7, 35.8, 35.4, 35.3, 34.7, 34.0, 29.6, 28.5, 27.6, 23.7, 22.6, 20.4, 17.3.

### Example 9

### Evaluation of Liver X Receptor Agonistic Activity

The liver X receptor agonistic activity of hypocholamide and hypocholaride was evaluated in a gene transactivation assay. See, e.g., Song, C. et al., Steroids, 2000, 65, 423-427.

Specifically, human embryonic kidney 293 cells were seeded into a 48-well culture plate at 10⁵ cells per well in a Dulbecco's modified Eagle's medium (DMEM) supplemented with 10% fetal bovine serum. After incubation for 24 hours, the cells were transfected by the calcium phosphate coprecipitation method with 250 ng of a pGL3/UREluc reporter gene that consisted of three copies of AGGTCAagccAGGTCA fused to nucleotides −56 to +109 of the human c-fos promoter in front of the firefly luciferase gene in the plasmid basic pGL3 (Promega, Madison, Wis.), 40 ng pSG5/hRXR_{α}, 40 ng pSG5/rUR or CMX/hliver X receptorα, 10 ng pSG5/hGrip1, 0.4 ng CMV/R-luc (transfection normalization reporter, Promega) and 250 ng carrier DNA per well. See, e.g., Janowski, B. A. et al., Nature, 1996, 383, 728-731; Song, C. et al., Endocrinology, 2000, 141, 4180-4184; Hong, H. et al., Proc. Natl. Acad. Sci. USA, 1996, 93, 4948-4952; and Amemiya-Kudo, M. et al., J. Biol. Chem., 2000, 275, 31078-31085.

After incubation for another 12 to 24 hours, the cells were washed with phosphate buffer saline and then refed with DMEM supplemented with 4% delipidated fetal bovine serum. An ethanol solution containing hypocholamide or hypocholaride was added in duplicate to the DMEM cell culture with the final concentration of hypocholamide of 1 to 10 μM and the final ethanol concentration of 0.2%. After incubation for another 24 to 48 hours, the cells were harvested and the luciferase activity was measured with a commercial kit (Promega Dual luciferase II) on a Monolight luminometer (Becton Dickenson, Mountain View, Calif.).

The results show that both hypocholamide and hypocholaride were unexpectedly potent agonists of liver X receptor alpha and liver X receptor beta (i.e., UR). For instance, hypocholaride had ED₅₀ values of 20 nM and 80 nM for liver X receptor alpha and liver X Receptor beta, respectively.

All references cited herein, whether in print, electronic, computer readable storage media or other form, are expressly incorporated by reference in their entirety, including but not limited to, abstracts, articles, journals, publications, texts, treatises, internet web sites, databases, patents, and patent publications.

A number of embodiments of the invention have been described. Nevertheless, it will be understood that various modifications may be made without departing from the spirit and scope of the invention. Accordingly, other embodiments are within claims.

What is claimed is:

1. A method for treating cancer, the method comprising administering to a subject in need thereof an effective amount of a Liver X receptor agonist having formula (I): 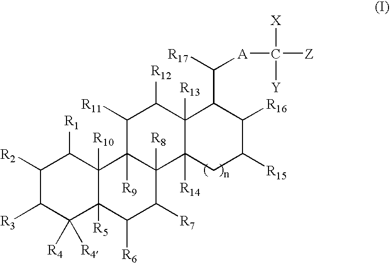
in which
each of R₁, R₂, R₃, R₄, R_{4′}, R₅, R₆, R₇, R₁₁, R₁₂, R₁₅, R₁₆, and R₁₇, independently, is hydrogen, halo, alkyl, haloalkyl, hydroxy, amino, carboxyl, oxo, sulfonic acid, or alkyl that is optionally inserted with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—; or R₃ and R₄ together, R₄ and R₅ together, R₅ and R₆ together, or R₆ and R₇ together are eliminated so that a C═C bond is formed between the carbons to which they are attached;
each of R₈, R₉, R₁₀, R₁₃, and R₁₄, independently, is hydrogen, halo, alkyl, haloalkyl, hydroxyalkyl, alkoxy, hydroxy, or amino;
n is 0, 1, or 2;
A is alkylene, alkenylene, or alkynylene; and
each of X, Y, and Z, independently, is alkyl, haloalkyl, —OR′, —SR′, —NR′R″, —N(OR′)R″, or —N(SR′)R″; or X and Y together are ═O, ═S, or ═NR′;
wherein each of R′ and R″, independently, is hydrogen, alkyl, or haloalkyl; or a salt thereof.

2. The method of claim 1, wherein each of R₅ and R₆, independently, is hydrogen, alkyl, haloalkyl, hydroxy, or amino.

3. The method of claim 2, wherein R₅ is H; and R₆ is hydroxy.

4. The method of claim 3, wherein X and Y together are ═O or ═S; and Z is —OR′, —SR′, —NR′R″, —N(OR′)R″, or —N(SR′)R″.

5. The method of claim 4, wherein X and Y together are ═O; and Z is —NR′R″, —N(OR′)R″, or —N(SR′)R″.

6. The method of claim 4, wherein each of R₁, R₂, R₃, R₄, R_{4′}, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

7. The method of claim 5, wherein each of R₁, R₂, R₃, R₄, R_{4′}, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

8. The method of claim 7, wherein each of R₁, R₂, R₄, R_{4′}, R₇, R₈, R₉, R₁₁, R₁₂, R₁₄, R₁₅, R₁₆, and R₁₇, independently, is hydrogen; R₃ is hydroxy; each of R₁₀ and R₁₃, independently, is alkyl; and A is alkylene.

9. The method of claim 3, wherein each of X, Y, and Z, independently, is alkyl, haloalkyl, —OR′, or —SR′.

10. The method of claim 9, wherein each of R₁, R₂, R₃, R₄, R_{4′}, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

11. The method of claim 10, wherein each of R₁, R₂, R₄, R_{4′}, R₇, R₈, R₉, R₁₁, R₁₂, R₁₄, R₁₅, R₁₆, and R₁₇, independently, is hydrogen; R₃ is hydroxy; each of R₁₀ and R₁₃, independently, is alkyl; and A is alkylene.

12. The method of claim 1, wherein R₅ and R₆ together are eliminated so that a C═C bond is formed between the carbons to which R₅ and R₆ are attached.

13. The method of claim 12, wherein X and Y together are ═O or ═S; and Z is —OR′, —SR′, —NR′R″, —N(OR′)R″, or —N(SR′)R″.

14. The method of claim 13, wherein X and Y together are ═O; and Z is —NR′R″, —N(OR′)R″, or —N(SR′)R″.

15. The method of claim 13, wherein each of R₁, R₂, R₃, R₄, R_{4′}, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

16. The method of claim 14, wherein each of R₁, R₂, R₃, R₄, R_{4′}, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

17. The method of claim 16, wherein each of R₁, R₂, R₄, R_{4′}, R₇, R₈, R₉, R₁₁, R₁₂, R₁₄, R₁₅, R₁₆, and R₁₇, independently, is hydrogen, halo, alkyl, haloalkyl, hydroxy, or amino; R₃ is hydroxy; each of R₁₀ and R₁₃, independently, is alkyl; n is 0; and A is alkylene.

18. The method of claim 12, wherein each of X, Y, and Z, independently, is alkyl, haloalkyl, —OR′, —SR′, —NR′R″, —N(OR′)R″, or —N(SR′)R″.

19. The method of claim 18, wherein each of R₁, R₂, R₃, R₄, R_{4′}, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

20. The method of claim 19, wherein R₃ is hydroxy; and each of R₁₀ and R₁₃, independently, is alkyl.

21. The method of claim 1, wherein X and Y together are ═O or ═S; and Z is —OR′, —SR′, —NR′R″, —N(OR′)R″, or —N(SR′)R″.

22. The method of claim 21, wherein X and Y together are ═O; and Z is —NR′R″, —N(OR′)R″, or —N(SR′)R″.

23. The method of claim 21, wherein each of R₁, R₂, R₃, R₄, R_{4′}, R₅, R₆, R₇, R₈, R₉, R₁₁, R₁₂, R₁₄, R₁₅, R₁₆, and R₁₇, independently, is hydrogen, halo, alkyl, haloalkyl hydroxy, or amino; each of R₁₀ and R₁₃, independently, is hydrogen, alkyl, or haloalkyl; n is 0; and A is alkylene.

24. The method of claim 22, wherein each of R₁, R₂, R₃, R₄, R_{4′}, R₅, R₆, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

25. The method of claim 24, each of R₁, R₂, R₄, R_{4′}, R₅, R₇, R₈, R₉, R₁₁, R₁₂, R₁₄, R₁₅, R₁₆, and R₁₇, independently, is hydrogen; each of R₃ and R₆, independently, is hydrogen or hydroxy; each of R₁₀ and R₁₃, independently, is alkyl; n is 0; and A is alkylene.

26. The method of claim 1, wherein each of X, Y, and Z, independently, is alkyl, haloalkyl, —OR′, —SR′, —NR′R″, —N(OR′)R″, or —N(SR′)R″.

27. The method of claim 26, wherein each of X, Y, and Z, independently, is alkyl, haloalkyl, —OR′, or —SR′.

28. The method of claim 26, wherein each of R₁, R₂, R₃, R₄, R_{4′}, R₅, R₆, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

29. The method of claim 27, wherein each of R₁, R₂, R₃, R₄, R₄, R₅, R₆, R₇, R₈, R₉, R₁₁, R₁₂, R₁₄, R₁₅, R₁₆, and R₁₇, independently, is hydrogen, halo, alkyl, haloalkyl, hydroxy, or amino; each of R₁₀ and R₁₃, independently, is hydrogen, alkyl, or haloalkyl; n is 0; and A is alkylene.

30. The method of claim 29, wherein each of R₁, R₂, R₄, R_{4′}, R₅, R₇, R₈, R₉, R₁₁, R₁₂, R₁₄, R₁₅, R₁₆, and R₁₇, independently, is hydrogen; each of R₃ and R₆, independently, is hydrogen or hydroxy; each of R₁₀ and R₁₃, independently, is alkyl.

31. The method of claim 1, wherein the compound is

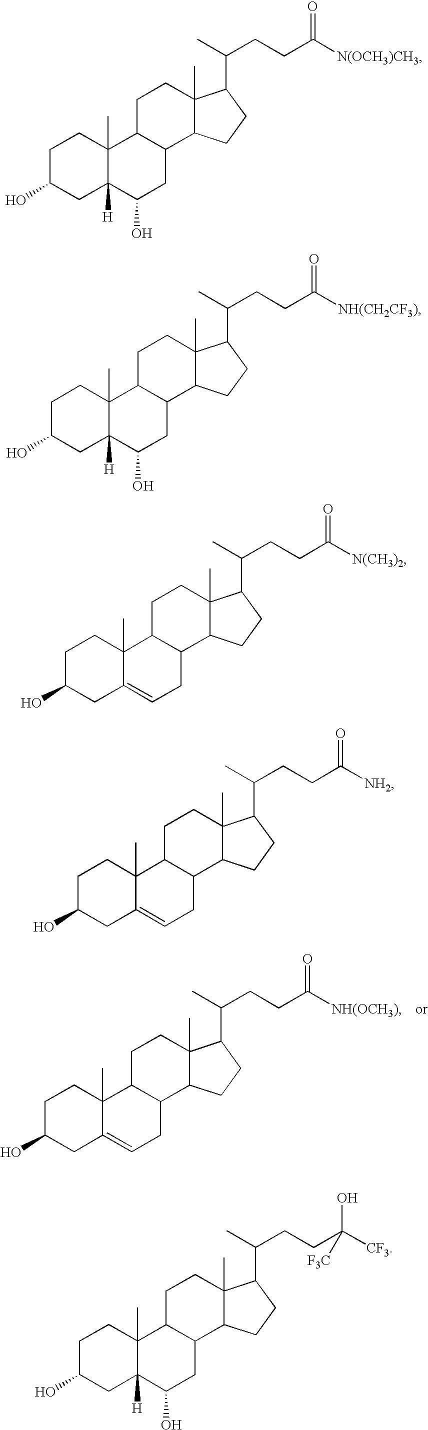

32. The method of claim 1, wherein the cancer is a sex hormone-dependent cancer.

33. The method of claim 32, wherein the sex hornone-dependent cancer is prostate cancer.

34. The method of claim 33, wherein the prostate cancer is an androgen-dependent prostate cancer.

35. The method of claim 33, wherein the prostate cancer is resistant to androgen deprivation and/or antiandrogen therapy.

36. The method of claim 35, wherein the prostate cancer is an androgen-independent prostate cancer.

37. The method of claim 36, wherein the androgen-independent prostate cancer is a hormone-refractory prostate cancer.

38. The method of claim 1, wherein the subject has at least one prostate cancer tumor that is resistant to androgen deprivation and/or antiandrogen therapy.

39. The method of claim 38, wherein the subject has at least one androgen-independent prostate cancer tumor.

40. The method of claim 38, wherein the subject is substantially free of androgen-dependent prostate cancer tumors.

41. The method of claim 1, wherein the Liver X receptor agonist is orally administered.

42. The method of claim 1, wherein the Liver X receptor is LXRα or LXRβ.

43. The method of claim 1, wherein the compound of formula (I) is administered with a pharmaceutically acceptable carrier or adjuvant.

44. The method of claim 1, wherein the salt is a pharmaceutically acceptable salt.

45. The method of claim 32, wherein the cancer is breast cancer.

46. The method of claim 1, wherein each of X, Y, and Z is, independently, haloalkyl or OR′.

47. The method of claim 46, wherein two of X, Y, and Z are, independently, haloalkyl, and the other is OR′.

48. The method of claim 47, wherein two of X, Y, and Z are, independently, haloalkyl, and the other is OH.

49. The method of claim 48, wherein two of X, Y, and Z are CF₃, and the other is hydroxy.

50. The method of claim 1, wherein
R₁, R₂, R₃, R₄, R₅, R₆, R₇, R₁₁, R₁₂, R₁₅, R₁₆, and R₁₇ are independently hydrogen, halo, alkyl, haloalkyl, hydroxy, amino, carboxyl, oxo, sulfonic acid, or alkyl that is optionally substituted at one or more positions with —NH—, —N(alkyl)-, —O—, —S—, —SO—, —SO₂—, —O—SO₂—, —SO₂—O—, —SO₃—O—, —CO—, —CO—O—, —O—CO—, —CO—NR′—, or —NR′—CO—;
R^{4′} is hydrogen;
Each of R₈, R₉, R₁₀, R₁₃, and R₁₄ is, independently, hydrogen, halo, alkyl, haloalkyl, hydroxyalkyl, alkoxy, hydroxy, or amino;
n is 0, 1,or 2;
A is alkylene, alkenylene, or alkynylene;
X, Y, and Z are independently alkyl, haloalkyl, —OR′, —SR′, —NR′R″, N(OR′)R″, or —N(SR′)R″; or X and Y together are ═O, ═S, or ═NR′; and
R′ and R″, are independently hydrogen, alkyl, or haloalkyl;
or a salt, an ester, an amide, an enantiomer, an isomer, a tautomer, a polymorph, a prodrug, or a derivative thereof.

51. The method of claim 50, wherein R₁, R₂, R₄, R₇, R₈, R₉, R₁₁, R₁₂, R₁₄, R₁₅, and R₁₆ are independently hydrogen; R₁₀, R₁₃, and R₂₀ are independently alkyl; n is 0; and A is alkylene.

52. The method of claim 51, wherein R₅ is hydrogen; and R₃ and R₆ are hydroxy.

53. The method of claim 52, wherein R₅ is beta-hydrogen; and R₃ and R₆ are alpha-hydroxy.

54. The method of claim 50, wherein R₅ is hydrogen; and R₃ and R₆ are hydroxy.

55. The method of claim 52, wherein R₅ is beta-hydrogen; and R₃ and R₆ are alpha-hydroxy.

56. The method of claim 50, wherein X, Y, and Z, are independently alkyl, haloalkyl, —OR′, or —SR′.

57. The method of claim 56, wherein R₁, R₂, R₄, R₇, R₈, R₉, R₁₁, R₁₂, R₁₄, R₁₅, and R₁₆ are hydrogen; R₁₀, R₁₃, and R₂₀ are alkyl; n is 0; and A is alkylene.

58. The method of claim 57, wherein R₅ is hydrogen; and R₃ and R₆ are hydroxy.

59. The method of claim 58, wherein R₅ is beta-hydrogen; and R₃ and R₆ are alpha-hydroxy.

60. The method of claim 56, wherein R₅ is hydrogen; and R₃ and R₆ are hydroxy.

61. The method of claim 60, wherein R₅ is beta-hydrogen; and R₃ and R₆ are alpha-hydroxy.

62. The method of claim 56, wherein X and Y together are ═O or ═S; and Z is —OR′, —SR′, —NR′R″, —N(OR′)R″, or —N(SR′)R″.

63. The method of claim 62, wherein R₁, R₂, R₄, R₇, R₈, R₉, R₁₁, R₁₂, R₁₄, R₁₅, and R₁₆ are hydrogen; R₁₀, R₁₃, and R₂₀ are alkyl; n is 0; and A is alkylene.

64. The method of claim 63, wherein R₅ is hydrogen; and R₃ and R₆ are hydroxy.

65. The method of claim 64, wherein R₅ is beta-hydrogen; and R₃ and R₆ are alpha-hydroxy.

66. The method of claim 62, wherein R₅ is hydrogen; and R₃ and R₆ are hydroxy.

67. The method of claim 66, wherein R₅ is beta-hydrogen; and R₃ and R₆ are alpha-hydroxy.

68. The method of claim 1, wherein the compound is

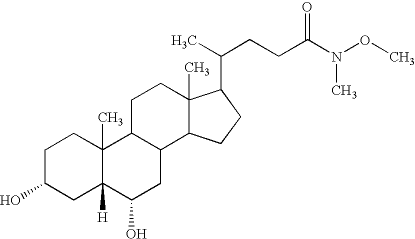

69. The method of claim 1, wherein the compound is [chemical structure]
